(19) United States
(12) Patent Application Publication
(10) Pub. No.: US 2023/0157987 A1
(43) Pub. Date: May 25, 2023

(54) COMBINATION OF CANNABIDIOL AND A PPAR AGONIST

(71) Applicants: Steven Robert Laviolette, London (CA); Marta De Felice, London (CA); Richard Rusiniak, Mississauga (CA); Paul Ramsay, Mississauga (CA)

(72) Inventors: Steven Robert Laviolette, London (CA); Marta De Felice, London (CA); Richard Rusiniak, Mississauga (CA); Paul Ramsay, Mississauga (CA)

(21) Appl. No.: 17/917,715

(22) PCT Filed: Apr. 9, 2021
(86) PCT No.: PCT/CA2021/050478

§ 371 Date: Oct. 7, 2022

Related U.S. Application Data
(60) Provisional application No. 63/007,529, filed on Apr. 9, 2020.

(51) Int. Cl. A61K 31/202; A61K 31/05

(52) U.S. Cl. CPC A61K 31/202; A61K 31/05

(57) ABSTRACT

The formulation includes CBD and a PPAR agonist. The PPAR agonist can be an omega-3 based PPAR agonist and more specifically can comprise substantially equal parts DHA and EPA.

### FIELD OF THE INVENTION

The invention relates to the field of CBD.

### BACKGROUND

CBD is believed by many to have therapeutic effects and it is known that relatively high doses of CBD decreases VTA dopamine activity. This inhibition of dopamine activity is related to the pharmacotherapeutic properties of CBD in producing anti-psychotic effects, anti-addictive effects, anti-anxiety effects and potential alleviation of traumatic memory disorders such as PTSD.

### SUMMARY

Forming one aspect of the invention is a formulation comprising CBD and a PPAR agonist.

According to another aspect, the PPAR agonist can be an omega-3 based PPAR agonist.

According to another aspect, the PPAR agonist can comprise substantially equal parts DHA and EPA.

Advantages, features and characteristics of the invention will become evident to persons of ordinary skill in the art upon review of the following description.

### BRIEF DESCRIPTION OF THE DRAWINGS

FIG. 1 shows VTA dopamine results from a first electrophysiology experiment

FIG. 2 shows VTA DA results from the first electrophysiology experiment

FIG. 3 shows VTA dopamine results from a second electrophysiology experiment

FIG. 4 shows VTA DA results from the second electrophysiology experiment

FIG. 5 shows VTA dopamine results from a third electrophysiology experiment

FIG. 6 shows VTA DA bursts from the third electrophysiology experiment

FIG. 7 shows apparatus used for elevated plus maze experiments

FIG. 8 shows open arm time spent results for a first elevated maze experiment

FIG. 9 shows open arm entry results for the first elevated maze experiment

FIG. 10 shows open arm time spent results for a second elevated maze experiment

FIG. 11 shows open arm entry results for the second elevated maze experiment

FIG. 12 shows open arm time spent results for a third elevated maze experiment

FIG. 13 shows open arm entry results for the third elevated maze experiment

FIG. 14 shows apparatus for use in a light dark box experiment

FIG. 15 shows time to first transition for a first light dark box experiment

FIG. 16 shows time to second transition for the first light dark box experiment

FIG. 17 shows transition number results for the first light dark box experiment

FIG. 18 shows time spent in light box results for the first light dark box experiment

FIG. 19 shows time to first transition results for a second light dark box experiment

FIG. 20 shows time to second transition for the second light dark box experiment

FIG. 21 shows transition number results for the second light dark box experiment

FIG. 22 shows time spent in light box results for the second light dark box experiment

FIG. 23 shows time to first transition results for a third light dark box experiment

FIG. 24 shows time to second transition for the third light dark box experiment

FIG. 25 shows transition number results for the third light dark box experiment

FIG. 26 shows time spent in light box results for the third light dark box experiment

FIG. 27 shows apparatus for use in open field experiments

FIG. 28 shows total ambulatory time results for a first open field experiment

FIG. 29 shows total ambulatory distance results for a first open field experiment

FIG. 30 shows center zone entry results for a further open field experiment

FIG. 31 shows center zone time results for the further open field experiment

FIG. 32 shows total ambulatory time results for the further open field experiment

FIG. 33 shows total ambulatory distance from the further open field experiment

FIG. 34 shows freezing time results for a first fear conditioning experiment

FIG. 35 shows freezing time results for a second fear conditioning experiment

FIG. 36 shows freezing time results for a third fear conditioning experiment

### DETAILED DESCRIPTION

Forming an embodiment of the invention is a formulation comprising CBD and a PPAR agonist, the PPAR agonist being an omega-3 based PPAR agonist comprising substantially equal parts DHA and EPA.

### Experimental

Numerous experiments were conducted.

Electrophysiology Experiments

Three experiments were run of this type. All included a vehicle. In addition, the components were as follows:

Experiment 1:

100 ng CBD; 100 ng CBD+100 ng T007; 100 ng T007

Experiment 2:

50 ng CBD; 0.5 nmol n-3; 50 ng CBD+0.5 nmol n-3

Experiment 3:

1 ng CBD; 0.25 nmol n-3; 1 ng CBD+0.25 nmol n-3; 1 ng CBD+0.25 nmol n-3+100 ng T007

In the above:
CBD=cannabidiol
T007=T0070907 (PPARg antagonist)
n-3=omega-3

The omega-3 formulation includes equal amounts of DHA and EPA. Two doses were used in this study. 0.5 nmol n-3 is made up of 0.5 nmol DHA and 0.5 nmol EPA. 0.25 nmol n-3 is made up of 0.25 nmol DHA and 0.25 nmol EPA

In this procedure, rats were anesthetized with urethane. An electrode was used to record the baseline frequency of dopamine neurons in the ventral tegmental area (VTA) for 5 minutes. After the baseline recording, combinations of CBD, omega-3, and T007 (a PPARg antagonist) were infused into the nucleus accumbens (NAc).

The post-infusion frequency of that neuron was compared to its pre-infusion frequencies.

Although the mechanisms are unclear, evidence suggests that CBD produces its therapeutic effects through the decrease of dopamine neuron activity in the VTA, as detailed below.

Experiment 1

One of the characteristics of dopamine cells is that they have a tonic mode of firing and a burst mode of firing. Therefore, both the frequency and burst changes following infusions were analyzed.

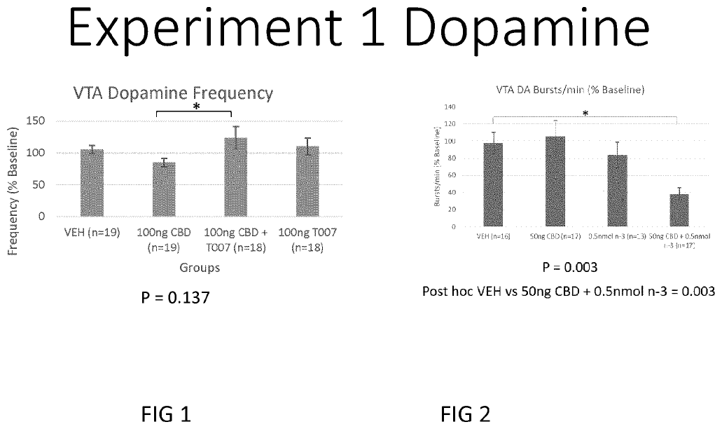

FIG. 1 looks at the frequency of VTA dopamine cells. The Y-axis is frequency (% Baseline). To clarify what this means: If one looks at the 100 ng CBD group, it shows about 85%. This means that after an intra-NAc infusion of 100 ng CBD, the dopamine cell would decrease in frequency about 15%.

FIG. 2 looking at difference in burst percentage. A burst is a group of signals occurring in a short period of time. Specifically, in this experiment, a burst is defined to be an event where 2 spikes occur within a timespan of 80 ms. Bursts do not occur very often so it is inaccurate to use the % baseline in the Y-axis (if 1 burst occurs during pre-infusion and 0 bursts during post-infusion, that's a 100% decrease although it is only 1 less burst event). As such, the number of bursts that occurred during pre-infusion was recording and the number of spikes that occurred in those bursts was determined. The number of those spikes occurring in those bursts was divided by the total number of spikes that occurred during the pre-infusion recording The same was done for the post-infusion recording and the difference between these 2 values was determined. Comparing FIG. 1 and FIG. 2, 100 ng CBD infusion caused a very slight decrease in bursts on average.

One-way ANOVA revealed that these groups are not significantly different.

However, it can be seen that 100 ng caused a decreasing trend in VTA dopamine frequency.

Experiment 2

In the second experiment, a lower dose of CBD (50 ng) was combined with omega-3 fatty acids.

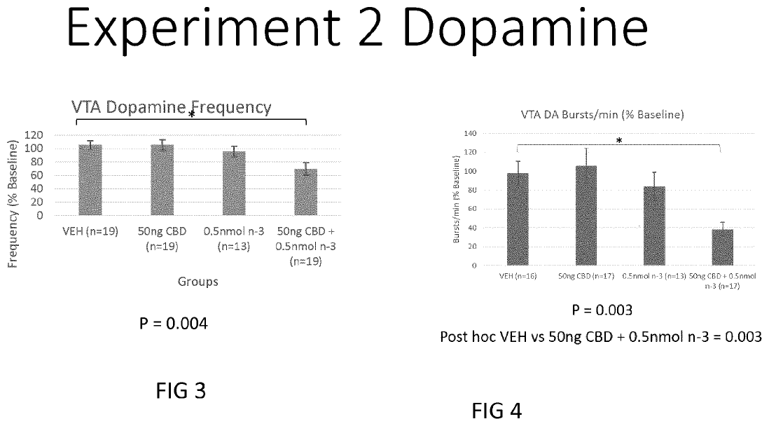

With reference to FIG. 3, one-way ANOVA revealed that the groups are significantly different. While 50 ng CBD and 0.5 nmol of omega-3 were ineffective by themselves, post-hoc analysis revealed that the combination of CBD and omega-3 caused a significant decrease in VTA dopamine activity.

With reference to FIG. 4, in terms of bursting, there were no significant differences.

Experiment 3

In this experiment, an attempt was made to replicate the results of experiment 2 with lower doses of CBD and omega-3.

With reference to FIG. 5, one-way ANOVA did not yield a significant effect (p=0.194). However, post-hoc analysis revealed that while 1 ng CBD and 0.25 nmol omega-3 were not different from vehicle, the combined 1 ng CBD+0.25 nmol omega-3 caused a significant decrease in VTA dopamine activity compared to vehicle. The addition of T007 to this combo blocked its effect which suggests that CBD and omega-3 may produce their effects through PPARg.

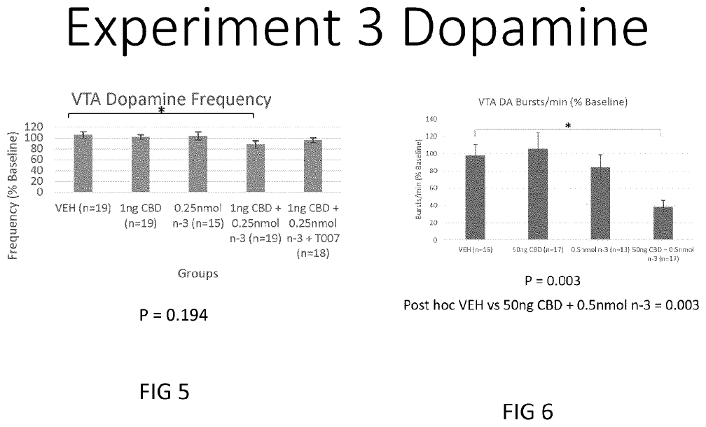

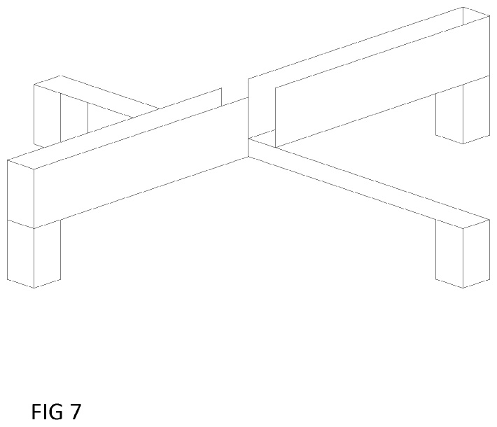

With reference to FIG. 6, no significant differences in bursts was observed.

Elevated Plus Maze Experiments

In these experiments, the following was measured:

1. Time in open arms

2. Entries into open arms

CBD produces its anxiolytic effects by decreasing VTA dopamine activity. We have just seen that CBD causes a slight decrease in VTA dopamine activity while combined CBD+omega-3 caused significant decreases.

An anxiolytic effect would therefor be expected when CBD and omega-3 are infused into the NAc of rats.

In these experiments, the apparatus of FIG. 7 was used.

As shown, the apparatus includes 2 open arms and 2 closed arms that form a plus shape. Rats feel more secure in closed spaces and thus it would be expected that more anxious rats would spend more time in the closed arms and less time on the open arms.

Before each test, the same doses of drugs used in the electrophysiology experiments were infused.

The rat was then placed in the middle of the apparatus, facing into the closed arms and recorded for 10 minutes.

The number of entries into the open arms and the total time spent in the open arms was recorded. Less anxious rats would be expected to make more entries into the open arms and spend more time there as well.

Experiment 1

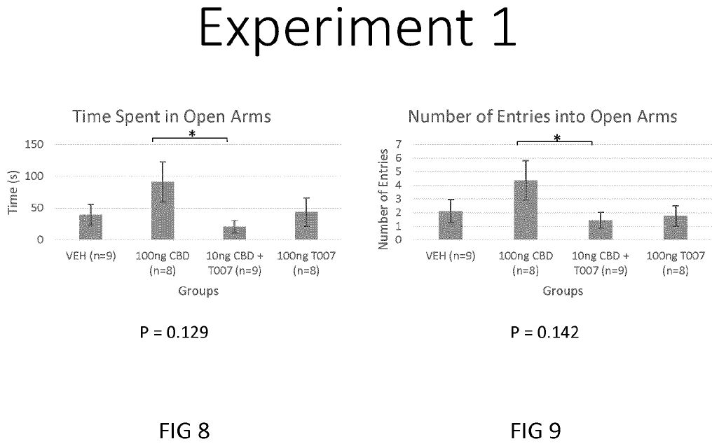

As shown in FIG. 8 and FIG. 9, no significant differences from one-way ANOVA in the time spent in open arms and the number of entries into open arms was observed. In the 100 ng CBD group however, we see a slight increase in the time spent in open arms and the number of entries into open arms which suggests a slight anxiolytic effect.

Experiment 2

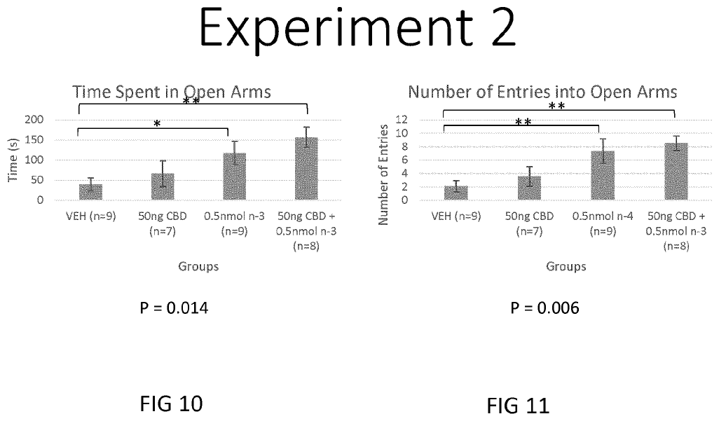

As shown by in FIG. 10 and FIG. 11, one-way ANOVA revealed a significant difference between groups for both the time spent in open arms and number of entries into open arms.

While 50 ng CBD was not effective, post-hoc analysis revealed that 0.5 nmol omega-3 significantly increased the time spent in open arms and entries into open arms as well.

The combination of 50 ng CBD and 0.5 nmol omega-3 had the same effect.

To determine if CBD and omega-3 would have a synergistic effect, the dose of CBD and omega-3 was decreased in experiment 3.

Experiment 3

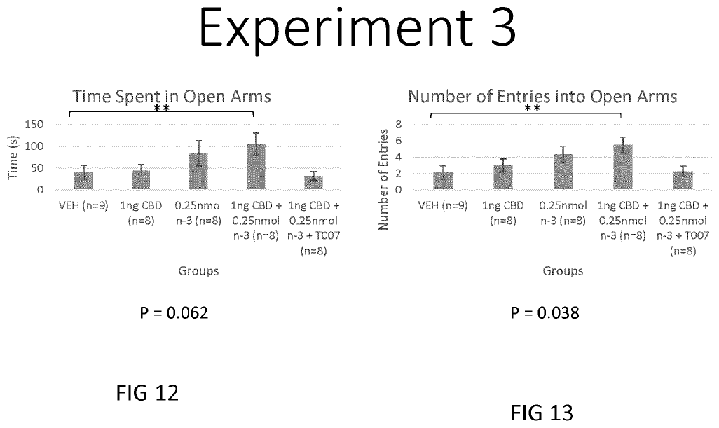

In FIG. 12, one-way ANOVA revealed that the groups are not significantly different. Both the 1 ng CBD and 0.25 nmol omega-3 doses were ineffective by themselves. However, post-hoc analysis revealed that the combination of 1 ng CBD+0.25 nmol omega-3 caused a significant increase in time spent in open arms. This effect was blocked by T007.

In FIG. 13, one-way ANOVA revealed that the groups are significantly different (p=0.038). Similar to the previous graph, post-hoc analysis revealed that the combination of 1 ng CBD with 0.25 nmol omega-3 significantly increased the number of entries into the open arms while 1 ng CBD and 0.25 nmol omega-3 alone were not effective. Again, T007 blocked the effects of the combined dose of CBD and omega-3 which support a conclusion that CBD and omega-3 produce their effects through PPARg.

Light Dark Box Experiment

In these experiments, the following was measured:

1. First transition to the dark box

2. Second transition to the light box

3. Total number of transitions

4. Time spent in the light box

The light-dark box is another anxiety test.

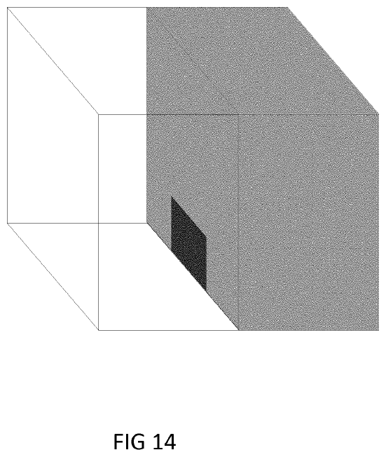

This experiment uses the apparatus made up of 2 boxes as shown in FIG. 14. One half of the box is open at the top and is brightly lit. There is an opening to the dark-box that is covered by a lid.

Rats would prefer to be in the dark box and would be anxious about going into the light box.

Following the infusion, the rate is placed in the light box facing away from the opening to the dark box. A 10 minute recording is made. During these 10 minutes, measurements are made of four things:

1. The time it takes for the rat to make the first transition into the dark box

2. The time it takes for the rat to make the second transition from the dark box back into the light box

3. Number of transitions between boxes

4. Time spent in light box

Therefore, it would be expected than less anxious rats would take a longer time to make the first transition into the dark box, spend less time to make the second transition into the light box, make more transitions between boxes, and spend more time in the light box.

Experiment 1

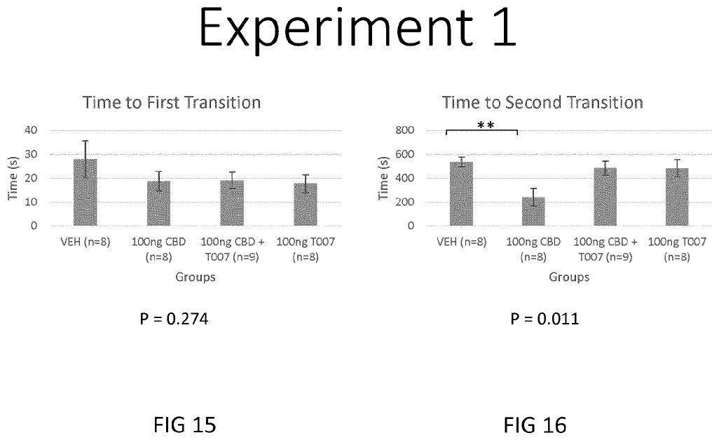

As shown in FIG. 15, there were no significant differences between any groups for the time to make the first transition into the dark box.

As shown in FIG. 16, one-way ANOVA revealed significant differences between groups. The post-hoc analysis revealed that 100 ng CBD significantly decreased the time to make the second transition back into the light box which is an anxiolytic effect. This effect was blocked by the addition of T007.

Experiment 2

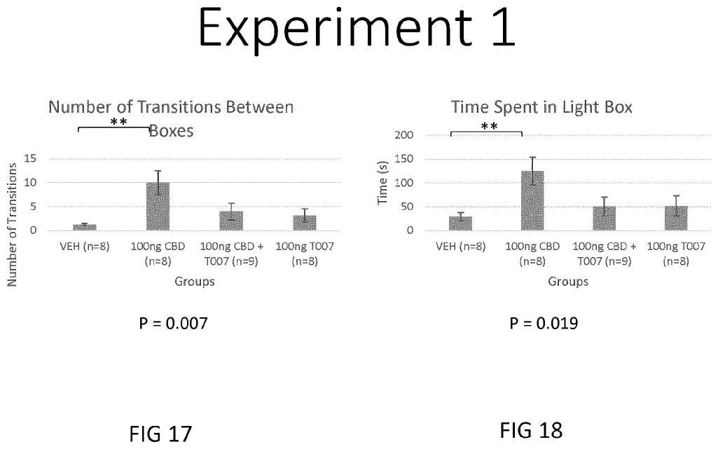

As shown in FIG. 17 and FIG. 18, 100 ng CBD significantly increased the number of transitions between boxes and the time spent in the light box while T007 blocked these effects.

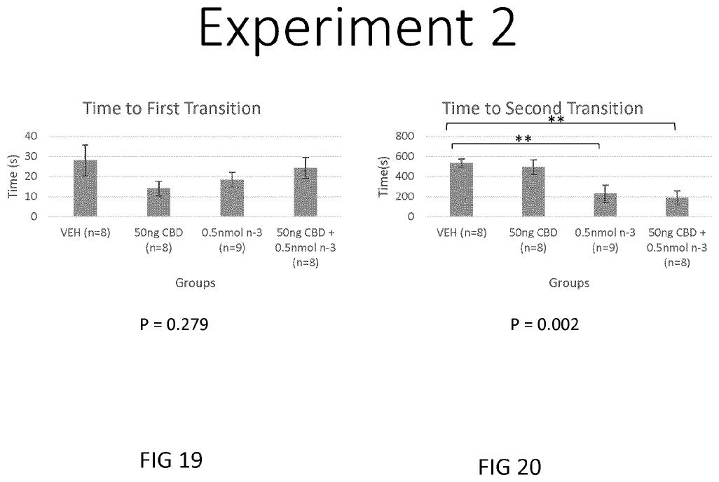

FIG. 19 shows no significant differences. With reference to FIG. 20, one-way anova revealed significant differences between groups. While 50 ng CBD was not effective, 0.5 nmol omega-3 by itself decreased the time to make the second transition. The combination of 50 ng CBD and 0.5 nmol omega-3 also caused a significant decrease.

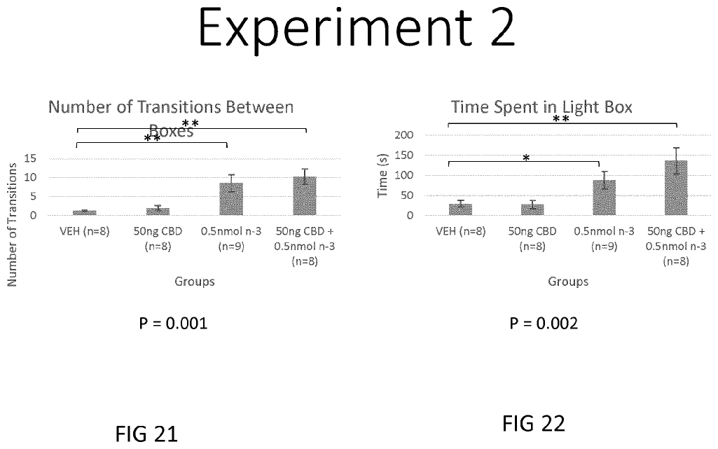

Similar to FIGS. 19 and 20, FIGS. 21 and 22 show that both the 0.5 nmol omega-3 alone and the combined 50 ng CBD+0.5 nmol omega-3 groups caused significant anxiolytic effects (increased the transitions between boxes and time spent in the light box).

The doses of CBD and omega-3 were decreased in experiment 3 to attempt to find a clear synergistic effect with CBD and omega-3.

Experiment 3

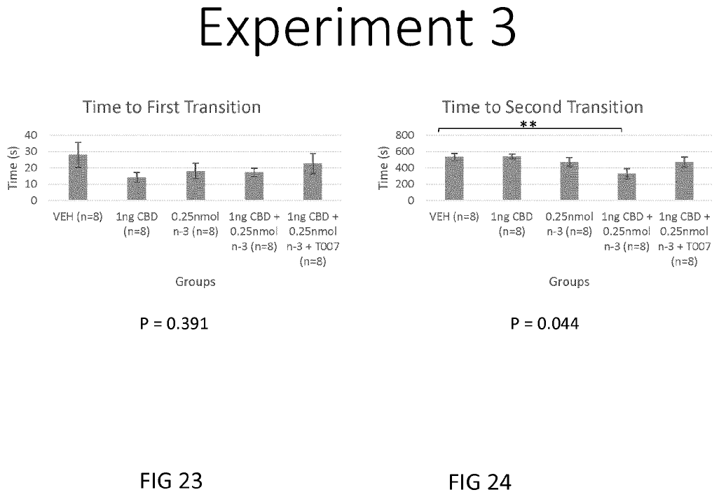

In FIG. 23, no significant effect was shown.

In FIG. 24, one-way ANOVA revealed significant differences between groups. As expected, 1 ng CBD and 0.25 nmol omega-3 alone were ineffective. However, the combination of 1 ng CBD and 0.25 nmol omega-3 significantly decreased the time to make the second transition. The addition of T007 blocked these effects.

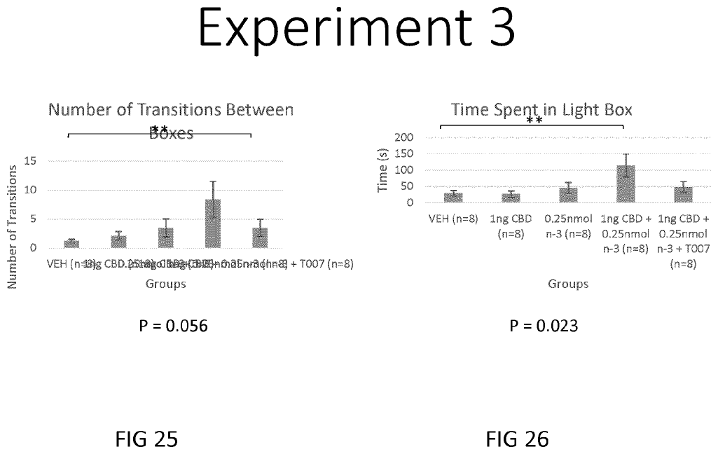

With reference to FIG. 25 and FIG. 26, 1 ng CBD and 0.25 nmol omega-3 alone are ineffective. Their combination, however, produced a significant anxiolytic effect (increased the number of transitions between boxes and the time spent in the light box).

Note that in FIG. 25, one-way ANOVA was not significant but the post-hoc analysis shows a significant effect of the combined 1 ng CBD+0.25 nmol omega-3 dose.

Open Field Experiments

In these experiments, the following was measured:

1. Time spent in center zone

2. Entry into center zone.

3. Total ambulatory time

4. Total ambulatory distance

Based on the results from FIGS. 25 and 26, it cannot be determined whether CBD and omega-3 are actually increasing locomotion or decreasing anxiety. In the light-dark box test for example, omega-3 and CBD may actually be increasing locomotion which would result in a shorter time to make the second transition, increased transitions between boxes, and therefore more time spent in the light box.

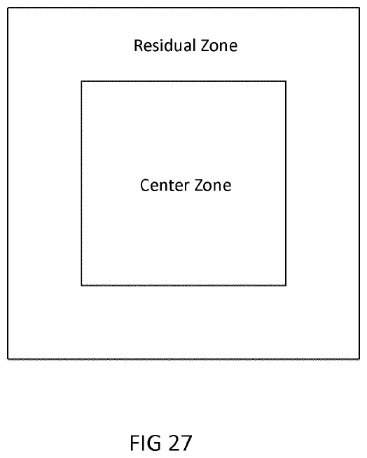

In the open field test, illustrated in FIG. 27, rats are placed in a box where they are free to move. Their ambulatory time and distance can be recorded to see if the drugs affect locomotion.

At the same time, we can also measure anxiety. Rats would prefer to be on the outer edges of the box so that one side of their body is covered by the wall of the box. They would feel anxious about being exposed in the center of the box. Therefore, less anxious rats would be expected to spend more time in the center zone.

Experiment 1

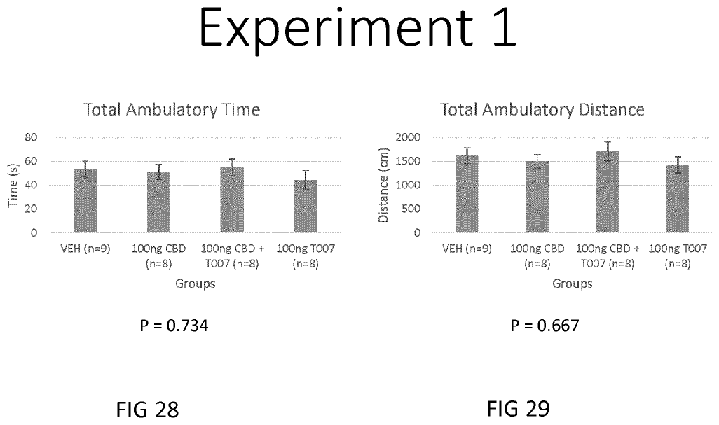

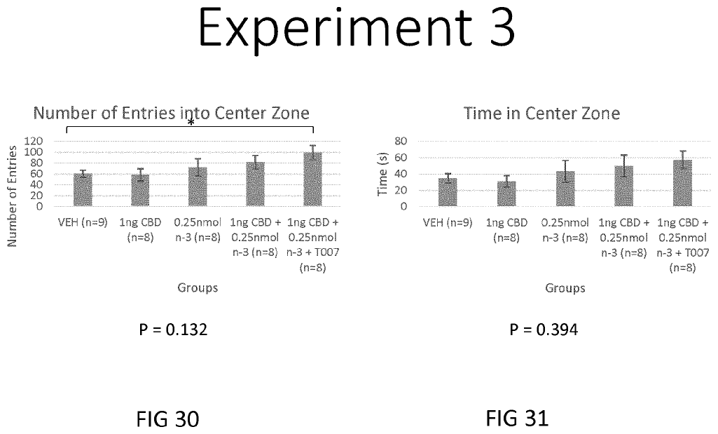

With reference to FIGS. 28 and 29, the drugs did not have an effect on locomotion (ambulatory time or ambulatory distance). With reference to FIGS. 30 and 31, one-way ANOVA did not reveal any significant differences between groups for entries into center zone and time spent in center zone.

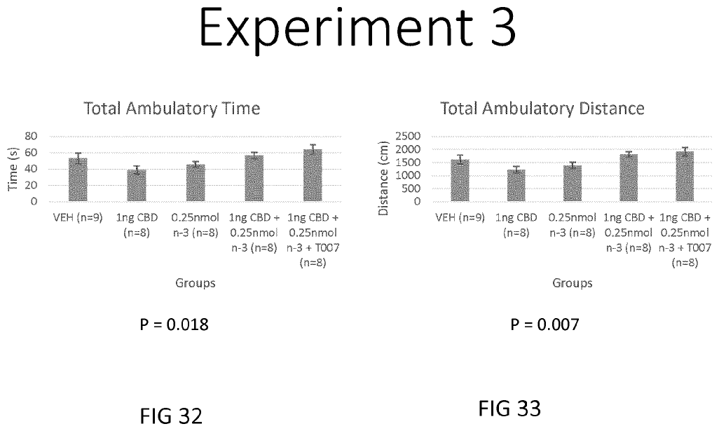

With reference to FIGS. 32 and 33, one-way ANOVA revealed significant differences for the total ambulatory time and total ambulatory distance. However, post-hoc analysis revealed that compared to vehicle, there were no significant differences.

Olfactory Fear Conditioning Experiments

The olfactory fear conditioning protocol is for measurement of the formation of fear memory. This protocol lasts three days. On day 1, the rat is habituated to two boxes (one with a striped background and one with a polka dot back ground).

On day 2, the rat receives a drug infusion and is placed into one of the boxes (previously assigned as the “shock box”). While in the box, the rat is exposed to 2 odours (peppermint and almond). The rat is exposed to one odour followed by the other odour 5 times. One of these odours were previously assigned as the “shock” odour (CS+) such that after exposure to the “shock” odour, the rat would receive a foot shock. There was no foot shock following exposure to the “safe” odour (CS−). On day 3, the rat was placed in the safe box. They were exposed to each odour (both CS+ and CS−) one at a time for 5 minutes. During the 5 minutes, freezing behaviour was recorded.

It would then be expected that vehicle rats would have associated the correct CS+ odour with the footshock and therefore demonstrate increased freezing during the CS+ odour exposure on day 3 compared to CS− exposure. It would be expected that CBD would block the formation of fear memory and the rats would freeze a similar amount to CS+ and CS−.

Experiment 1

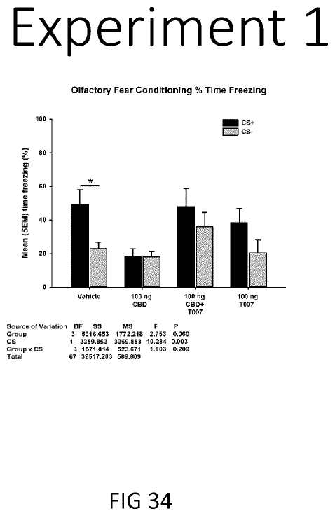

2-way repeated measures ANOVA was performed for the three experiments. With reference to FIG. 34, there was no significant interaction effect by group x CS (p=0.209). While the VEH group had a significant difference between CS+ and CS− freezing time, the other groups did not have a significant difference.

Experiment 2

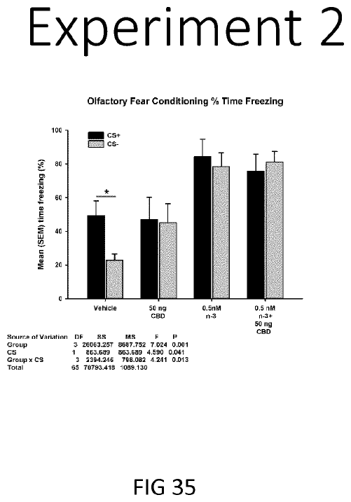

In experiment 2, there was a significant interaction effect (p=0.013) by groups and CS. While the VEH had a significant difference between CS+ and CS− freezing time, the other groups did not which suggests that the other groups are blocking fear memory formation, as shown by FIG. 35.

Experiment 3

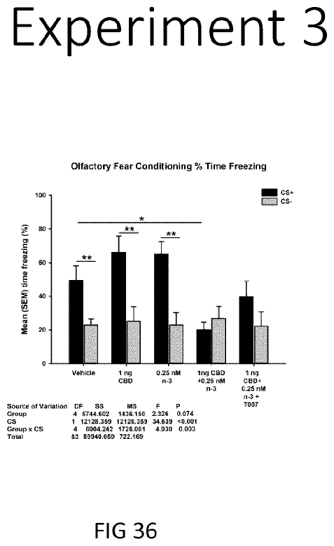

In experiment 3, as shown in FIG. 36, there was a significant interaction effect (p=0.003) by groups and CS. While the 1 ng CBD and 0.25 nmol omega-3 are not different from vehicle, the combined 1 ng+0.25 nmol omega-3 group is significantly different from vehicle which suggests a synergistic effect.

Note in vehicle, 1 ng CBD, and 0.25 nmol omega-3, there was a significant difference between the freezing time to CS+ and CS− which suggests that those groups have working fear memory formation. The combined 1 ng CBD+0.25 nmol omega-3 dose blocks the formation of fear memory. This effect seems to be blocked slightly by T007.

### ANALYSIS

Succinctly, the aforementioned experimentation reveals that the co-administration of a PPAR agonist allowed for CBD dosage to be significantly reduced while maintaining CBD's inhibitory effects on DA neuron activity states as well as potentiating CBD's ability to produce anti-anxiety and anti-PTSD effects in pre-clinical models of these disorders in rodents. Further, the combination of CBD+Omega-3 inhibited ‘bursting rates’. This is important because bursting of DA neurons is linked to DA dysregulation in mental health disorders like addiction, schizophrenia and anxiety. This suggests that the combination may be more effective than CBD alone in treating a variety of mental health disorders including addiction, anxiety, schizophrenia: basically, anything that can normalize abnormal DA firing/bursting rates is likely good for these disorders.

### ADVANTAGES

It will be apparent that this has significant advantage, since CBD is relatively costly. As well, high doses of CBD creates huge bolus concentrations, which creates the potential for side-effects.

### DOSAGES

Whereas in this document, specific dosage regimes are described, it will be appreciated by persons of ordinary skill that the dosages mentioned have been proven to be useful in the context of rats and in experimental conditions; routine experimentation will be required to modify the dosages for human use.

### VARIATIONS

Whereas a specific PPAR agonist is described, namely, a combination of DHA and EPA in substantially equal amounts, it is contemplated that other PPARA agonists might be useful, including but not limited to: Honokiol, magnolol, Echinacea purpurea (L.), Panax ginseng and 10-hydroxy-octadecanoic acid.

Accordingly the invention should be understood to be limited only by the accompanying claims, purposively construed.

The contents of U.S. Provisional Patent Application Ser. No. 63/007,529 are incorporated herein by reference.

1. A formulation comprising CBD and a PPAR agonist.

2. A formulation according to claim 1, wherein the PPAR agonist is an omega-3 based PPAR agonist.

3. A formulation according to claim 2, wherein the PPAR agonist comprises substantially equal parts DHA and EPA.
